(19) United States
(12) Patent Application Publication
(10) Pub. No.: US 2001/0053204 A1
(43) Pub. Date: Dec. 20, 2001

(54) Method and apparatus for relative calibration of a mobile X-ray C-arm and an external pose tracking system

(76) Inventors: Nassir Navab, Plainsboro, NJ (US); Matthias Mitschke, Nuernberg (DE)

Correspondence Address: Siemens Corporation, Intellectual Property Department, 186 Wood Avenue South, Iselin, NJ, 08830, US

(21) Appl. No.: 09/779,064

(22) Filed: Feb. 8, 2001

Related U.S. Application Data
(60) Provisional application No. 60/181,481, filed on Feb. 10, 2000.

(51) Int. Cl. A61B 6/08

(52) U.S. Cl. 378/205; 378/162; 378/207

(57) ABSTRACT

An external tracking system for use three dimensional (3D) reconstruction with X-ray C-arm systems comprises a set of marker or tool plates and a sensor system. It measures the 6D pose (position and orientation) of these tool plates relative to the sensor. The tool plate of such a tracking system is rigidly attached to the X-ray source frame of the C-arm system. In order to use an external tracking system for the recovery of projection geometry for 3D tomographic reconstruction, the relations of the external sensor coordinate systems to patient/world and X-ray coordinate systems have to be determined. Previously the transformation from tool plate to X-ray coordinate system has been determined based on CAD drawings and a calibration of the focal point of the X-ray system.

Reference is hereby made to co-pending Provisional U.S. patent application Ser. No. 60/181,481 entitled “Relative calibration of a mobile X-RAY C-ARM and an external pose tracking system”, filed in the names of Navab and Mitschke on Feb. 10, 2000 and whereof the disclosure is hereby incorporated herein by reference.

The present application relates to X-ray C-arm calibration and, more particularly, to the relative calibration of a mobile C-arm and an external pose tracking system.

In prior art work on three dimensional (3D) reconstruction using X-ray C-arm systems, the geometry calibration is generally done offline, using an X-ray calibration phantom, when the motion of the C-arm is reproducible. See, for example, U.S. Pat. Nos. 5,822,396 and 5,835,563. If the motion of the C-arm is not reproducible, the geometry calibration is done online using a (charge coupled device) CCD camera, or an external tracking system.. See, for example, U.S. Pat. No. 5,923,727. The disclosure of the said U.S. Pat. Nos. 5,822,396 and 5,835,563 is herein incorporated by reference to the extent that it is not incompatible with the present invention.

The external tracking system utilized in such a procedure comprises a set of marker or tool plates and a sensor system. The system measures the 6D pose (position and orientation) of these tool plates relative to the sensor. The tool plate of such a tracking system is rigidly attached to the X-ray source frame of the C-arm system.

In order to use an external tracking system for the recovery of projection geometry for 3D tomographic reconstruction, the relationships of the external sensor coordinate systems to patient/world and X-ray coordinate systems have to be determined. In the prior art, the transformation from tool plate to X-ray coordinate system has typically been determined based on CAD drawings and a calibration of the focal point of the X-ray system.

Such methods in accordance with the prior art are subject to problems of manufacturing precision and a weak calibration of the projection geometry of the X-ray system.

In accordance with an aspect of the present invention, a method for calibration comprises calibrating the transformation from tool plate to X-ray coordinate system as well as the less important sensor to world coordinate transformation from a set of C-arm positions with known X-ray projection geometry.

The invention will be more fully understood from the detailed description of preferred embodiments which follows, in conjunction with the Drawing in which

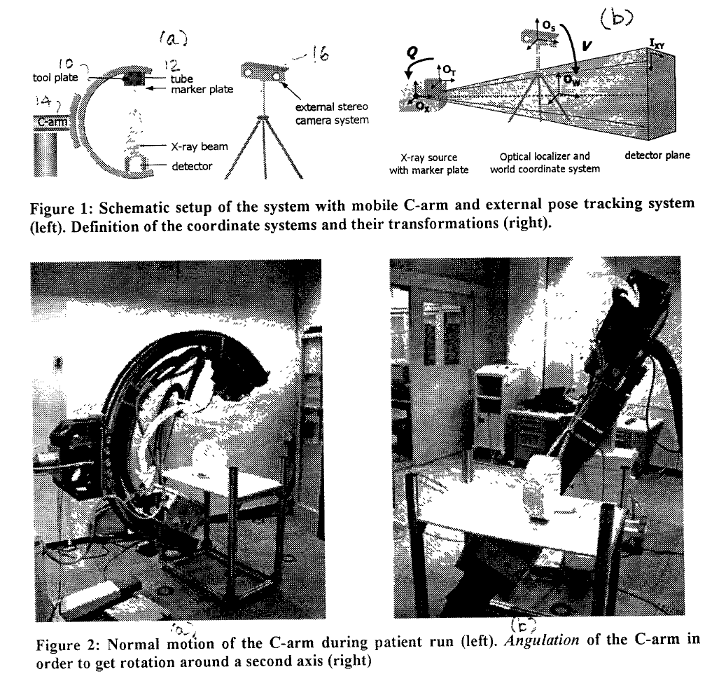

FIG. 1a and 1b shows a setup in accordance with the principles of the invention;

FIG. 2a shows the normal motion of a C-arm during a patient run; and

FIG. 2b shows angulation of the C-arm in order to obtain rotation around a second axis.

FIG. 1a shows a setup in accordance with the principles of the invention. The tool plate 10 of the pose tracking system is rigidly attached to the frame of the X-ray source 12 of the C-arm system 14. The external tracking system—here shown by way of example as a stereo camera system 16—tracks the position and orientation of the attached tool plate in its local coordinate system.

FIG. 1b shows the definition of the different coordinate systems and the two coordinate transformations Q and V that have to be determined.

The calibration task to be solved herein is equivalent to the well-known problem known as hand-eye calibration in the fields of robotics and computer vision as described in technical articles. See, for example, R. Y. Tsai and R. K. Lenz, A new technique for fully autonomous and efficient {3D } robotics hand/eye calibration. IEEE Transactions on Robotics and Automation, 5(3):345-358, 1989; R. Horaud and F. Dornaika, Hand-eye calibration. International Journal of Robotics Research, 14(3):195-210, 1995; K. Daniilidis. Hand-eye calibration using dual quaternions. International Journal of Robotics Research, 18(3):286-298, 1999.

By way of analogous example, a sensor—typically a CCD camera—is mounted on the gripper of a robot. In order to be able to position the robot's gripper such that the CCD camera gets into a well-defined position relative to its target, the relation between gripper and camera coordinate system has to be determined beforehand. The robot's motion is defined by the pose, that is, the position and orientation, of the gripper relative to its local world coordinate system. With the mounted CCD camera, the position and orientation of a target object are measured, defined in another world coordinate system, relative to the camera's own coordinate system.

The motion performed by the robot in the analogous example used, has to be transformed into a motion in the coordinate system of the attached sensor. Assuming that the robot itself is not moving, there are two rigid transformations that have to be determined during this calibration procedure: “gripper” to “sensor” and “robot world” to “target object world”.

In the setup in accordance with the present invention, the C-arm takes the position of the attached sensor whereas the tracking system becomes the equivalent of the robot. It is assumed that the sensor of the external tracking system is not moved during the calibration procedure such that there remains a constant relation to the patient/world coordinate system.

The mathematical details of this procedure can be found in the literature as, for example, in the above cited technical publications and need not be fully set forth herein.

With the two unknown transformations, Q and V, estimated, the goal then becomes one of recovering the projection geometry of the X-ray C-arm system from the pose information, position and orientation, which the tracking system provides. The two unknown transformations are Q: O_{T} to O_{X}, and V:O_{S} to O_{W,} using the symbols defined below.

With regard to the different coordinate systems involved, and with reference to FIG. 1(b), O_{W} is a world coordinate system and O_{X} is the X-ray coordinate system. I_{X} is the image plane. O_{S} is the coordinate system of exterior stereo camera system 16, and coordinate system O_{T} is attached to marker plate 12. The transformation L_{I} denotes the transformation from O_{S} to O_{T} for image frame i.

For an arbitrary image frame the transformation from the world (O_{w}) to X-ray coordinate system (Oₓ)—denoted by Eᵢ—using the pose information of the tracking system Lᵢ is described by

Eᵢ=Q·Lᵢ·V⁻¹ (1)

This transformation followed by the projection onto the image plane I_{X}—described by the intrinsic parameters Aᵢ—defines the projection geometry Pᵢ. As shown in the above-cited technical publications, instead of Q, the slightly different transformation S is first estimated. This new unknown transformation S is defined as

Q:=E_{ref}·S (2)

where E_{ref} is assumed to be a known transformation from world to X-ray coordinate system (extrinsic geometry) for a reference frame. This leads to the following equation for the X-ray projection geometry

Pᵢ=A_{ref}·E_{ref}·S·Lᵢ·V⁻¹

Pᵢ=P_{ref}·S·LᵢV⁻¹ (3)

Instead of an additional estimation of the necessary transformation E_{ref} a reference projection matrix P_{ref} is used for the recovery of projection geometry.

According to the mathematical theory, 3 positions of the C-arm are sufficient to compute the unknown transformations. By incorporating more positions, the accuracy can be increased and the transformations are solved using a linear least square technique. The only requirement on the minimum number of 3 positions is that the resulting 2 motions must be rotations about different axes. In the setup in accordance with the invention, the C-arm only rotates about one axis in a patient run (about 190° degrees of rotation), which results in between 50 and 100 images. In order to fulfill the calibration requirement of two different rotations a different kind of rotation has to be applied to the C-arm. FIG. 2 shows the two different rotations, the standard rotation during a normal patient run (FIG. 2a) and the special rotation “angulation” (FIG. 2b). The calibration results will be best if the two rotation axes are orthogonal to each other, and the amount of motion between the image frames is as large as possible.

As explained above, the theory of hand-eye calibration requires the sensor of the pose tracker to stay in a fixed relation to the patient/world coordinate system. Therefore the pose tracking system would have to be re-calibrated whenever it is moved around in the operating room. This would mean a limitation of the present method.

As described in equation (3) the final X-ray geometry needed for 3D reconstruction for frame i is determined from the pose information provided by the pose tracker as follows:

Pᵢ=P_{ref}·S·LᵢV⁻¹

A change of the sensor to patient/world transformation V only changes the position of the object in the reconstructed volume. The patient/world coordinate system depends on the position of the X-ray calibration phantom during the calibration process. Often a re-normalization of the set of determined projection matrices is performed such that the rotation axis of the C-arm during the patient run becomes one of the coordinate axes of the reconstructed volume and the origin is placed in the center of the reconstruction volume.

If it be assumed that the set of projection matrices is normalized before the 3D reconstruction step the determination of the transformation V becomes less important. All projection matrices are post-multiplied by a re-normalization transformation N that depends on the whole set of projection matrices. However V is chosen, a normalizing transformation N can be determined such that the set of projection matrices is transformed into the pre-defined coordinate system.

While the invention has been described by way of exemplary embodiments, it will be understood by one of skill in the art to which it pertains that various modifications and amendments made to it without departing from the spirit of the invention and that such changes and modifications are intended to be included within the scope of the claims following.

What is claimed is:

1. A method for relative calibration of a mobile X-ray C-arm system including an X-ray source and an external pose system, comprising:
coupling a set of marker or tool plates rigidly to an X-ray source; measuring the 6-dimensional (6D) pose (position and orientation) of said tool plates relative to a sensor system;
utilizing data derived from said sensor system for calculating a transformation from a coordinate system attached to said tool plates to a coordinate system attached to said X-ray source;
utilizing data derived from said sensor system for calculating a transformation from a coordinate system attached to said sensor system to a coordinate system attached to a predefined world coordinate system; and
utilizing results obtained in the preceding two steps for calculating the projection parameters of said X-ray C-arm system.

2. A method for relative calibration of a mobile X-ray C-arm system in accordance with claim 1 wherein said step for calculating a transformation from a coordinate system attached to said tool plates to a coordinate system attached to said X-ray source utilizes data based on CAD drawings and a calibration of the focal point of said X-ray system.

3. A method for relative calibration of a mobile X-ray C-arm system in accordance with claim 2 including the step of:
calculating the transformation, denoted by Eᵢ, for an arbitrary image frame, from said world coordinate system (O_{w}) to said X-ray coordinate system (Oₓ) utilizing data for a tracking system, including pose information of tracking system Lᵢ derived from said sensor system, in accordance with the equation
Eᵢ=Q·Lᵢ·V⁻¹ (1)

4. A method for relative calibration of a mobile X-ray C-arm system in accordance with claim 3 including, following said step of calculating the transformation from said world coordinate system (O_{w}) to said X-ray coordinate system (Oₓ), the step of:
projecting said arbitrary image frame onto an image plane I_{X}, defined by intrinsic parameters Aᵢ-Aₛ.

5. A method for relative calibration of a mobile X-ray C-arm system in accordance with claim 3 wherein said step of calculating the transformation, denoted by E₁ comprises a step of calculating a transformation S in accordance with the equation
Q:=E_{ref}·S (2)
where E_{ref} is assumed to be a known transformation from world to X-ray coordinate system for a reference frame.

6. A method for relative calibration of a mobile X-ray C-arm system in accordance with claim 5 including a step for calculating said X-ray projection geometry in accordance with the equations:
Pᵢ=A_{ref}·E_{ref}·S·Lᵢ·V⁻¹
Pᵢ=P_{ref}·S·Lᵢ·V⁻¹
wherein a reference projection matrix P_{ref} is used for the recovery of projection geometry.

7. An external tracking system for use three dimensional (3D) reconstruction with an X-ray C-arm system, including an X-ray source, comprises:
a set of marker or tool plates rigidly coupled to an X-ray source;
a sensor system for measuring the 6-dimensional (6D) pose (position and orientation) of said tool plates relative to said sensor system;
means utilizing data derived from said sensor system for calculating a transformation from a coordinate system attached to said tool plates to a coordinate system attached to said X-ray source;
means utilizing data derived from said sensor system for calculating a transformation from a coordinate system attached to said sensor system to a coordinate system attached to a predefined world coordinate system; and
means utilizing results obtained in the preceding two steps for calculating the projection parameters of said X-ray C-arm system.

8. An external tracking system in accordance with claim 7 wherein said means for calculating a transformation from a coordinate system attached to said tool plates to a coordinate system attached to said X-ray source utilizes data based on CAD drawings and a calibration of the focal point of said X-ray system.
